SEQUENCE LISTING (4 sequences)

SEQ ID NO: 1 - protein, 13 aa, Artificial Sequence
  engineered from known peptides
HGVSGHGQHGVHG

SEQ ID NO: 2 - protein, 12 aa, Artificial Sequence
  engineered from known peptides
GVSGHGQHGVHG

SEQ ID NO: 3 - protein, 8 aa, Artificial Sequence
  engineered from known peptides
SGHGQHGV

SEQ ID NO: 4 - protein, 10 aa, Artificial Sequence
  engineered from known peptides
VSGHGQHGVH

(19) United States
(12) Patent Application Publication
(10) Pub. No.: US 2025/0073270 A1
(43) Pub. Date: Mar. 6, 2025

(54) COMPOSITION FOR PREVENTING OR TREATING CANCER, COMPRISING NK CELLS CULTURED USING ALLOFERON

(71) Applicant: NK MEDICS INC., Seoul (KR)

(72) Inventors: Gwang Jin YOO, Seongnam-si (KR); Woon Young SEO, Seoul (KR)

(21) Appl. No.: 18/727,440

(22) PCT Filed: Jan. 28, 2022
(86) PCT No.: PCT/KR2022/001590

§ 371 Date: Jul. 9, 2024

(30) Foreign Application Priority Data
Jan. 28, 2022 (KR) 10-2022-0012883

(51) Int. Cl. A61K 35/17; A61P 35/00; C12N 5/0783

(52) U.S. Cl. CPC A61K 35/17; A61P 35/00; C12N 5/0646; C12N 2501/998

(57) ABSTRACT

The present invention relates to a composition for preventing or treating cancer, comprising NK cells cultured in the presence of alloferon, wherein the NK cells cultured using alloferon have excellent cancer-killing properties. Further, when alloferon and NK cells are administered together, the cancer prevention or killing effect can be further increased.

### TECHNICAL FIELD

The present invention relates to a composition for preventing or treating cancer, comprising NK cells cultured in the presence of alloferon, wherein the NK cells cultured using alloferon have excellent cancer-killing properties, and thus can be usefully used in the prevention or treatment of cancer.

### BACKGROUND ART

Several natural components containing substances from animal and plant tissues, including insects, that can stimulate the effectiveness of the immune system, are known. Among them, alloferon is a linear peptide having a molecular weight of 1265 daltons derived from the immune material of infected flies. According to various studies so far, alloferon has been found to exhibit a wide range of antiviral, anticancer, anti-inflammatory and anti-allergic effects through immune modulating effects. Alloferon is rapidly absorbed into the body and remains stably for a long period of time, thereby maximizing its efficacy. In particular, alloferon is marketed as a Class I new drug in the classification of non-toxic drugs.

Meanwhile, NK cells are one of the immune cells included in lymphocytes, and are called ‘natural killer (NK) cells’ due to the characteristic that they have the ability to kill external enemies from the moment they are produced. NK cells act widely within the body, and when abnormal cells such as cancer cells and virus-infected cells are discovered, they can first attack them independently. This independence and immediate effect are the biggest characteristics of NK cells. Since NK cells do not have an antigen-antibody response, they can directly attack abnormal cells freely and flexibly, and are attracting attention as a cell therapy agent because the capability is remarkable even among immune cells that attack cancer cells.

Korean Patent Publication No. 10-0394864 discloses the use of alloferon as an anticancer agent. However, it does not disclose that its use as a culture material for NK cells can have an effect on increasing the growth rate of NK cells or on NK cell activity.

### DETAILED DESCRIPTION OF THE INVENTION

### Technical Problem

One aspect provides a composition for preventing or treating cancer comprising NK cells cultured in the presence of a polypeptide represented by the amino acid sequence of SEQ ID NOs: 1-4 or a combination thereof.

In one embodiment, the NK cells are cultured in a medium containing 6 mg/L to 40 mg/L of the polypeptide or a combination thereof.

In another embodiment, the NK cells are cultured in a medium containing a polypeptide represented by any one of the amino acid sequences of SEQ ID NOs: 1-4 or a combination thereof; and IL-2, IL-12, IL-15, OKT-3, or a combination thereof.

In yet another embodiment, the cancer is a solid cancer.

In yet another embodiment, the solid cancer is selected from the group consisting of biliary tract cancer, lung cancer, breast cancer, prostate cancer, melanoma, and pancreatic cancer.

In yet another embodiment, the NK cells are contained at a density of 1×10⁵ cell/ml or more.

Another aspect provides a composition for preventing or treating cancer comprising a polypeptide represented by the amino acid sequence of SEQ ID NOs: 1-4 or a combination thereof; and NK cells.

In one embodiment, the composition containing alloferon and NK cells contains 10 μg or more of the polypeptide represented by the amino acid sequence of SEQ ID NOs: 1 to 4 or a combination thereof based on 1×10⁶ cells of NK cells.

### Technical Solution

According to one aspect, there is provided a composition for preventing or treating cancer comprising NK cells cultured in the presence of a polypeptide represented by the amino acid sequence of SEQ ID NOs: 1-4 or a combination thereof.

In the present specification, each polypeptide is an alloferon, and specifically, the polypeptide represented by SEQ ID NO: 1 may be named alloferon 1, the polypeptide represented by SEQ ID NO: 2 may be named alloferon 2, the polypeptide represented by SEQ ID NO: 3 may be named alloferon 3, and the polypeptide represented by SEQ ID NO: 4, may be named alloferon 4.

The polypeptide may be a combination of one or more selected from the group consisting of alloferon 1, alloferon 2, alloferon 3, and alloferon 4. Specifically, in the composition for culturing NK cells according to one embodiment, the polypeptide may include alloferon 1, alloferon 2, alloferon 3, and alloferon 4 as individual substances, and may include a combination of alloferons 1 and 2, a combination of alloferons 1 and 3, and a combination of alloferons 1 and 4, a combination of alloferons 2 and 3, a combination of alloferons 2 and 4, a combination of alloferons 3 and 4, or a combination of alloferons 1, 2, 3, and 4.

NK cells contained in the composition for preventing or treating cancer of the present invention are cultured in the presence of a composition for culturing NK cells that increases the survival rate and activity of NK cells, wherein the composition for culturing NK cells may further include one or more components selected from the group consisting of IL-2 (interleukin-2), IL-12 (interleukin-12), IL-15 (interleukin-15), and OKT-3 (Anti-CD3 antibody). Specifically, the composition for culturing NK cells may, in addition to IL-2, further include one or more components selected from the group consisting of IL-12, IL-15, and OKT-3. Here, IL-2 may be included in an amount of 0.5 mg/L to 5 mg/L, 0.5 mg/L to 4 mg/L, 1 mg/L to 3 mg/L, 1 mg/L to 2.5 mg/L, 1.5 mg/L to 2.5 mg/L, or 2 mg/L relative to the total volume of the culture medium. IL-12 may be included in an amount of 0.5 mg/L to 5 mg/L, 0.5 mg/L to 4 mg/L, 1 mg/L to 3 mg/L, 1 mg/L to 2.5 mg/L, 1.5 mg/L to 2.5 mg/L, or 2 mg/L relative to the total volume of the culture medium. IL-15 may be included in an amount of 5 mg/L to 15 mg/L, 7 mg/L to 12 mg/L, 8 mg/L to 11 mg/L, or 10 mg/L relative to the total volume of the culture medium. OKT-3 may be included in an amount of 10 mg/L to 30 g/L, 12 mg/L to 28 mg/L, 15 mg/L to 25 mg/L, 18 mg/L to 22 mg/L, 19 mg/L to 21 mg/L, or 20 mg/L relative to the total volume of the culture medium. The composition for culturing NK cells may include IL-2, but may not include IL-12, IL-15, and OKT-3. If the composition includes the combination of IL-2 and alloferon, it can remarkably increase the culture efficiency of NK cells, and therefore, it may be advantageous in reducing costs and minimizing side effects due to the inclusion of unnecessary components. However, in order to solve problems such as culture stability, IL-12, IL-15, or OKT-3 can be added to IL-2 and alloferon, or the amount can be appropriately increased or decreased.

In addition, the composition for culturing NK cells may further include any one or more components selected from the group consisting of FBS or autologous plasma, human serum albumin, insulin, and transferrin. The FBS or autologous plasma may be included in an amount of 1 v/v % to 20 v/v %, 5 v/v % to 15 v/v %, 8 v/v % to 12 v/v %, or 10 v/v % relative to the total volume of the culture medium. The human serum albumin may be included in an amount of 100 mg/L to 3,000 mg/L, 500 mg/L to 2,500 mg/L, 1,000 mg/L to 2,500 mg/L, 1,500 mg/L to 2,500 mg/L, or 1,800 mg/L to 2,200 mg/L, or 2,000 mg/L relative to the total volume of the culture medium. The transferrin may be included in an amount of 5 mg/L to 15 mg/L, 7 mg/L to 12 mg/L, 8 mg/L to 11 mg/L, or 10 mg/L relative to the total volume of the culture medium. The composition for culturing NK cells includes a medium commonly known as a basic medium, for example, RPMI 1640 medium, but is not limited thereto. The composition for culturing NK cells includes the polypeptide in an amount of 6 mg/L to 40 mg/L, 8 mg/L to 30 mg/L, 8 mg/L to 25 mg/L, 8 mg/L to 20 mg/L, 8 mg/L to 15 mg/L, 8 mg/L to 12 mg/L, or 10 mg/L.

The content of each component of the composition for culturing NK cells may be an amount suitable for culturing NK cells at a density of 1×10⁴ cells/ml to 1×10⁶ cells/ml, or 1×10⁵ cells/ml.

NK cells contained in the composition for preventing or treating cancer of the present invention are cultured with the composition for culturing NK cells as described above, whereby compared to before culture, the expression level of NKp30 may be 45% or more, preferably 55% or more, the expression level of NKp44 may be 50% or more, preferably 60% or more, the expression level of NKp46 may be 65% or more, preferably 80% or more, and the expression level of perforin may be 90% or more, preferably 99% or more. In addition, when cultured in a medium containing the composition for culturing NK cells of the present invention, the amount of IFN-gamma secreted may be increased by 3 times or more, 4 times or more, 5 times or more, or 6 times compared to when cultured in a medium not containing alloferon.

NK cells contained in the composition for preventing or treating cancer of the present invention may have a density of 1×10⁵ cell/ml or more, 2.5×10⁵ cell/ml or more, 5×10⁵ cell/ml or more, 1×10⁶ cell/ml or more, 5×10⁶ cell/ml or more, 1×10⁷ cell/ml or more, or 5×10⁷ cell/ml or more.

In another aspect, there is provided a composition for preventing or treating cancer comprising a polypeptide represented by the amino acid sequence of SEQ ID NOs: 1 to 4 or a combination thereof; and NK cells.

The composition for preventing or treating cancer comprising alloferon may include alloferon in an amount of 10 μg or more, 20 μg or more, 30 μg or more, 40 μg or more, or 45 μg or more based on 2.5×10⁶ cells of NK cells. The upper limit of the alloferon content may be 1,000 μg or less, 800 μg or less, 500 μg or less, 100 μg or less, or 80 μg or less. The composition for preventing or treating cancer, more specifically, the composition for preventing or treating cancer may include alloferon in an amount of 10 μg to 100 μg, 20 μg to 80 μg, 30 μg to 70 μg, 40 μg to 60 μg, or 50 μg.

In yet another aspect, there is provided a method of preventing or treating cancer, comprising administering the composition for preventing or treating cancer according to one embodiment to a subject in need thereof.

The composition for preventing or treating cancer of the present invention can be used as a pharmaceutical composition for preventing or treating cancer comprising natural killer cells as an active ingredient.

The pharmaceutical composition means a “cellular therapeutic agent”. As used herein, the term “cellular therapeutic agent” refers to cells and tissues prepared by isolation from an individual, culture and special operations, and means a pharmaceutical product (US FDA regulations) which is used for the purposes of treatment, diagnosis and prevention and which is obtained through a series of actions, including growing and screening living autologous or allogenic cells in vitro in order to restore the structure and function of the cells or changing the biological characteristics of cells by any other methods.

As used herein, the term “prevention” refers to any action that inhibits or delays the onset of cancer diseases by administration of the pharmaceutical composition. Further, as used herein, the term “treatment” refers to any action that ameliorates or beneficially change symptoms of cancer diseases by administration of the pharmaceutical composition.

In the present invention, the pharmaceutical composition may be characterized by being in the form of capsules, tablets, granules, injections, ointments, powders, or beverages, and the pharmaceutical composition may be characterized by being targeted to humans.

The pharmaceutical composition may be formulated in the form of oral preparations such as powders, granules, capsules, tablets, and aqueous suspensions, preparations for external use, suppositories, and sterile injectable solutions, respectively, according to conventional methods, and used. However, the pharmaceutical composition is not limited thereto. The pharmaceutical composition of the present invention may comprise a pharmaceutically acceptable carrier. As the pharmaceutically acceptable carrier, a binder, a glidant, a disintegrant, an excipient, a solubilizer, a dispersant, a stabilizer, a suspending agent, a pigment, a flavor, and the like may be used for oral administration; a buffer, a preserving agent, a pain-relieving agent, a solubilizer, an isotonic agent, a stabilizer, and the like may be used in admixture for injections; and a base, an excipient, a lubricant, a preserving agent, and the like may be used for topical administration. The preparations of the pharmaceutical composition of the present invention may be prepared in various ways by being mixed with the pharmaceutically acceptable carrier as described above. For example, for oral administration, the pharmaceutical composition may be formulated in the form of tablets, troches, capsules, elixirs, suspensions, syrups, wafers, or the like. For injections, the pharmaceutical composition may be formulated in the form of unit dosage ampoules or multiple dosage forms. Alternatively, the pharmaceutical composition may be formulated into solutions, suspensions, tablets, capsules, sustained-release preparations, or the like.

Meanwhile, as examples of carriers, excipients or diluents suitable for making preparations, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, or the like may be used. In addition, a filler, an anti-coagulant, a lubricant, a wetting agent, a fragrance, an emulsifier, a preservative, and the like may further be included.

The route of administration of the pharmaceutical composition of the present invention includes, but is not limited to, oral, intravenous, intramuscular, intraarterial, intramedullary, intradural, intracardiac, transdermal, subcutaneous, intraperitoneal, intranasal, intestinal, topical, sublingual, or rectal route. Parenteral administration is preferred.

As used herein, the term “parenteral” includes subcutaneous, intradermal, intravenous, intramuscular, intraarticular, intrabursal, intrasternal, intradural, intralesional, and intracranial injection or infusion techniques.

The pharmaceutical composition of the present invention may vary depending on a variety of factors, including activity of a certain compound used, the patient's age, body weight, general health status, sex, diet, time of administration, route of administration, rate of excretion, drug combination, and severity of a certain disease to be prevented or treated. A dose of the pharmaceutical composition may vary depending on the patient's condition, body weight, severity of disease, drug form, route of administration, and duration, and may be appropriately selected by those skilled in the art. The pharmaceutical composition may be administered in an amount of 0.0001 to 50 mg/kg or 0.001 to 50 mg/kg, per day. Administration may be made once a day or several times a day. The dose is not intended to limit the scope of the invention in any way.

### Advantageous Effects

A composition for preventing or treating cancer according to one aspect has excellent viability by culturing NK cells in the presence of alloferon, and includes the effectively activated NK cells, thereby exhibiting excellent cancer-killing properties. Therefore, even by using a small amount of NK cells, excellent cancer prevention and treatment effects can be obtained.

In addition, a composition for preventing or treating cancer according to another aspect includes alloferon and NK cells together, and thus can still exhibit remarkable cancer prevention and treatment effects even after administration.

### BRIEF DESCRIPTION OF THE DRAWINGS

FIG. 1 is a schematic diagram of a xenograft model production and efficacy evaluation method.

FIG. 2 is a graph showing changes in tumor size measured over time in a biliary tract cancer transplantation model.

FIG. 3 is a graph showing the relative amount of size change at the time of final biopsy in a biliary tract cancer transplantation model.

FIG. 4 is a graph showing changes in tumor size measured over time in a lung cancer transplantation model.

FIG. 5 is a graph showing the relative amount of size change at the time of final biopsy in a lung cancer transplantation model.

FIG. 6 is a graph showing changes in tumor size measured over time in a breast cancer transplantation model.

FIG. 7 is a graph showing the relative amount of size change at the time of final biopsy in a breast cancer transplantation model.

FIG. 8 is a graph showing changes in tumor size measured over time in a prostate cancer transplantation model.

FIG. 9 is a graph showing the relative amount of size change at the time of final biopsy in a prostate cancer transplantation model.

FIG. 10 is a graph showing changes in tumor size measured over time in a melanoma transplantation model.

FIG. 11 is a graph showing the relative amount of size change at final biopsy in a melanoma transplantation model.

FIG. 12 is a graph showing changes in tumor size measured over time in a pancreatic cancer transplantation model.

FIG. 13 is a graph showing the relative amount of size change at the time of final biopsy in a pancreatic cancer transplantation model.

FIG. 14 is a graph confirming the tumor growth rate according to alloferon treatment conditions in a lung cancer xenograft model.

FIG. 15 is a photograph showing the change in tumor size appearing at the time of final biopsy in a biliary tract cancer transplantation model.

FIG. 16 is a photograph showing the change in tumor size appearing at the time of final biopsy in a lung cancer transplantation model.

FIG. 17 is a photograph showing the change in tumor size appearing at the time of final biopsy in a breast cancer transplantation model.

FIG. 18 is a photograph showing the change in tumor size appearing at the time of final biopsy in a prostate cancer transplantation model.

FIG. 19 is a photograph showing the change in tumor size appearing at the final biopsy in a melanoma transplantation model.

FIG. 20 is a photograph showing the change in tumor size appearing at the final biopsy in a pancreatic cancer transplantation model.

### DETAILED DESCRIPTION OF THE EMBODIMENTS

Hereinafter, the present invention will be described in more detail with reference to Experimental Examples. The following Experimental Examples are only for illustrating the present invention in more detail, and according to the gist of the present invention, the scope of the present invention is not limited by the Experimental Examples.

### Preparation Example 1. Culture of NK cells in the Presence of Alloferon

### 1-1. Obtainment of NK Cells

30 ml of blood was collected from a human blood donor, RosetteSep NK enrichment antibody cocktail (50 ul/ml) was added thereto, and then diluted 1:1 v/v with phosphate buffer solution (PBS). The blood was centrifuged at 1600 rpm for 20 minutes using a density gradient centrifugation (Ficoll-Plaque) to separate the cell layer containing NK cells. The process of washing and centrifuging this with PBS was repeated twice. Then, the number of cells was counted by staining with trypan blue, and the average number of cells obtained was 1.6˜3.0×10⁵ cell/ml, and the cells were cultured in NK cell culture medium.

### 1-2. Preparation of NK Cell Culture Medium

The NK cell culture medium used was a combination of various substances that were conventionally known to be necessary for NK culture. Specifically, RPMI1640 was used as the basic medium, to which 10% (v/v) FBS or autologous plasma, human serum albumin 2,000 mg/L, recombinant human insulin 10 mg/L, recombinant human transferrin 10 mg/L, recombinant human interleukin 2 (IL-2) 2 mg/L, recombinant human interleukin 12 (IL-12) 2 mg/L, recombinant human interleukin 15 (IL-15) 10 mg/L and anti-CD3 antibody (OKT-3) 20 mg/L were mixed. This was used as the basic NK cell culture medium, to which alloferons 1 to 4 was further mixed and used at 0.1 to 100 mg/L as needed. The amino acid sequence of each alloferon is shown in Table 1 below.

TABLE 1

| | |
|---|---|
| Alloferon 1 (SEQ ID NO: 1) | HGVSGHGQHGVHG |
| Alloferon 2 (SEQ ID NO: 2) | GVSGHGQHGVHG |
| Alloferon 3 (SEQ ID NO: 3) | SGHGQHGV |
| Alloferon 4 (SEQ ID NO: 4) | VSGHGQHGVH |

### 1-3. NK Cell Culture

NK cells were cultured in the NK cell culture medium prepared according to the Preparation Example. As a result, when cultured in a medium containing alloferons 1 to 4, respectively, clusters containing very small numbers of NK cells were present in whole blood cells before culture, but it was observed that the size and number of these clusters increased after culture, confirming that it is suitable as a medium condition for culturing NK cells.

### Test Example 1: Evaluation of Toxicity of NK Cells in Mouse Body

It was evaluated whether the prepared NK cells had toxicity in mice. Specifically, NCr athymic nude mice (BALB/cSlc-nu/nu) (Charles River Laboratories) were manipulated and raised in a microisolator cage according to the Laboratory Practice for Nonclinical Laboratory Studies (21CFR Part 58, Food and Drug Administration, United States of America) (Apr. 1, 2015). In order to evaluate dose-dependent safety and toxicity, NK cells obtained as above were prepared in three different doses. The low-dose injection was 5×10⁵ cells/animal, equivalent to a dose of 2×10⁷ cells/kg in humans, and medium and high dose injections were designated as 1×10⁶ cells/animal and 1×10⁷ cells/animal, respectively. After that, the NK cells amplified by NK medium+alloferon 1 were used as Example 1, and a group in which this was administered and a group in which NK cells isolated from human serum were divided and used as a control group. Male and female mice were combined and a total of 50 animals (at around 6 weeks, the weight of each male and female was equivalent to 18.7˜22.5 g and 16.1˜18.8 g, respectively) were injected intravenously twice a week at 3-week intervals for 27 weeks. NK cells were injected intravenously into the tail vein a total of 18 times.

As a result, it could be confirmed that even when NK cells were injected at the maximum dose, it was safe without any fatal reaction. In addition, the observation of internal organs in all NK cell-treated groups showed that any type of toxicity could not observed in relation to cell administration. Further, no special changes were observed in T and B lymphocytes, indicating that the NK cells according to the present invention did not cause immune-related side effects.

### Test Example 2: Evaluation of Anticancer Effect in Xenograft Model

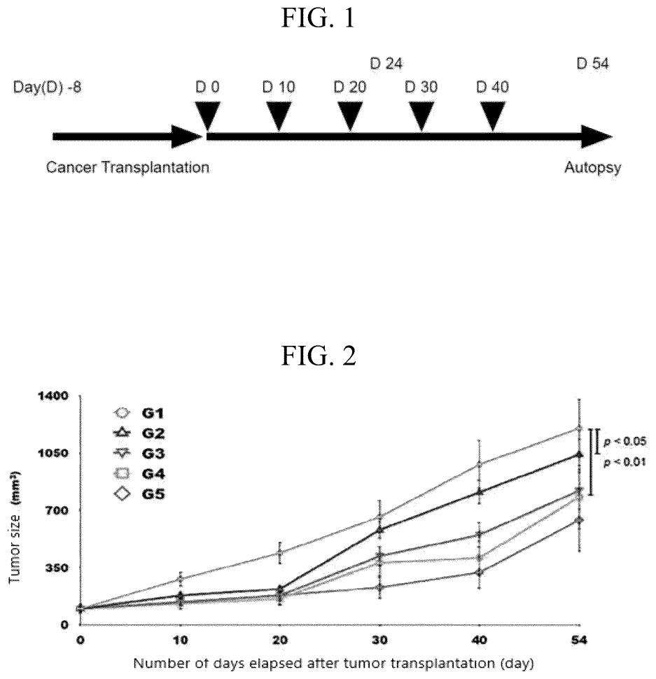

The carcinomas used were biliary tract cancer (HuCCT-1), lung cancer (H460), breast cancer (MDA-MB-231), prostate cancer (PC3), melanoma (B16F10), and pancreatic cancer (Capan-2). The schematic diagram of the xenograft model production and efficacy evaluation method followed the schedule as shown in FIG. 1, but the number of days and methods were slightly adjusted according to the nature of each carcinoma. The number of cells administered and the type of administered cells for each group are shown in Table 2 below.

TABLE 2

| Group | Cell Count | |
|---|---|---|
| Name | (Cells/0.2 ml) | Description |
| G1 | 0 | 0.2 ml physiological saline solution treated |
| | | group |
| G2 | 5 × 10⁵ | Cells cultured under NK medium + alloferon |
| | | 1 (10 mg/L) conditions (low dose) |
| G3 | 1 × 10⁶ | Cells cultured under NK medium + alloferon |
| | | 1 (10 mg/L) conditions (medium dose) |
| G4 | 1 × 10⁷ | Cells cultured under NK medium + alloferon |
| | | 1 (10 mg/L) conditions (high dose) |
| G5 | CDDP + Gem | Positive control group |
| G6 | 1 × 10⁶ | Cells cultured in general medium |
| G7 | 1 × 10⁶ | Cells cultured in NK medium (without |
| | | alloferon) conditions |

### 2-1. Bile Duct Cancer

NCr athymic nude mice (BALB/cSlc-nu/nu) (Charles River Laboratories) were manipulated and raised in a microisolator cage according to the Laboratory Practice for Nonclinical Laboratory Studies (21CFR Part 58, Food and Drug Administration, United States of America) (Apr. 1, 2015). HuCCT1 cells (5×10⁶ cells/0.2 mL) were subcutaneously injected into each group (G1˜G5) consisting of 10 nude mice, and subjected to transplantation and engraftment: G1, physiological saline solution (negative control group); G2˜G4, NK cell injection of Example 1; and G5, gemcitabine+cisplatin (Gem+CDDP) (positive control group). In each group, eight mice with well-engrafted tumors with a volume of 84˜119 mm³ (body weight range of 19.3˜20.5 g) were selected and then subjected to medical treatments. NK cells were injected intravenously five times at 10-day intervals into nude mice bearing HuCCT-1 tumors. As a positive control group, gemcitabine (Gem) and cis-diamineplatinum (II) dichloride (CDDP) were administered at doses of 120 mg/kg and 3 mg/kg, respectively. When injecting NK cells, three groups of mice were administered at different doses as follows: G2-G4: G2, low dose (5×10⁵ cell/animal); G3, medium dose (1×10⁶ cells/animal); G4, high dose (1×10⁷ cells/animal). G1 and G5 correspond to a negative control group (physiological saline) and a positive control group (CDDP+Gem), respectively. A disposable syringe (26G, 1 ml) was used for cell injection. The injection volume was set to 0.2 ml, and 14 days after the last injection, mice bearing tumors were euthanized and then evaluated.

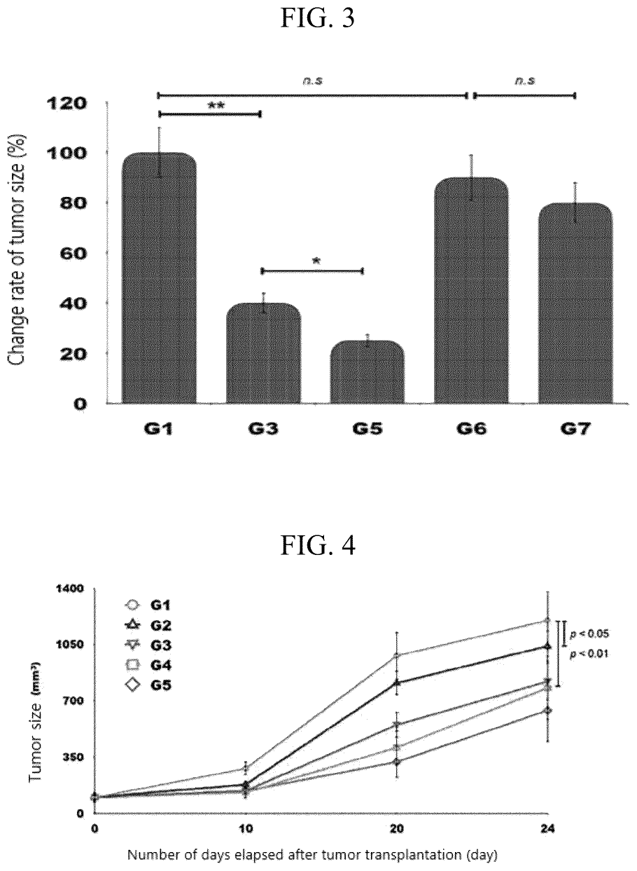

The results were evaluated by the level of apoptosis for each individual, and shown in Table 3 below, the tumor size according to the number of days elapsed after tumor transplantation is shown graphically in FIG. 2, and the relative amount of size change at the time of final biopsy in the transplantation model is shown in FIG. 3. For the measurement of the relative amount of size change at the time of final biopsy, the amount of change in G1 was considered as 100(%) among the average amount of size change during the experiment period for each group (G1, G3, G5, G6, G7), and the amount of change in each group was converted into a relative value, and shown in a graph. Average size change=Dt−D0/D0 (Dt=size on the last date that autopsy was performed)

Surprisingly, the group in which NK cells cultured using alloferon was administered showed a more excellent tumor growth inhibition effect than the positive control group. In particular, even when administered at the same number of cells, NK cells cultured using alloferon (G3) showed a remarkably superior tumor growth inhibition effect as compared to cells cultured in general medium (G6) or cells cultured in general NK medium (G7).

TABLE 3

| | | Proportion of | Level of apoptosis | | | |
|---|---|---|---|---|---|---|
| | N | death signal | for each individual | | | |
| Group | number | positive cells (%) | − | + | ++ | +++ |
| G1 | 8 | 4.3 | 8 | 0 | 0 | 0 |
| | | (SD = 3.7) | | | | |
| G2 | 8 | 11.7 | 1 | 2 | 5 | 0 |
| | | (SD = 5.2) | | | | |
| G3 | 8 | 24.6 | 1 | 3 | 3 | 1 |
| | | (SD = 9.4) | | | | |
| G4 | 8 | 32.7 | 0 | 2 | 3 | 3 |
| | | (SD = 10.1) | | | | |
| G5 | 8 | 22.8 | 0 | 3 | 4 | 1 |
| | | (SD = 8.7) | | | | |

−: No change, +: Weak, ++: Moderate, +++: Severe

### 2-2. Lung Cancer

The same procedure as Test Example 2-1 was performed, but H460 cells (5×10⁶ cells/0.2 mL) were subcutaneously injected into each group (G1˜G5) consisting of 10 nude mice and subjected to transplantation and engraftment. After that, the lung cancer xenograft model was performed in the same manner as in Test Example 2-1, but the injection volume was 0.2 ml. In the case of lung cancer, the 30-day survival rate after transplantation was low, and so the mice were sacrificed on the 24th, four days after D20. Mice bearing tumor were euthanized 4 days after the last injection, and then evaluated.

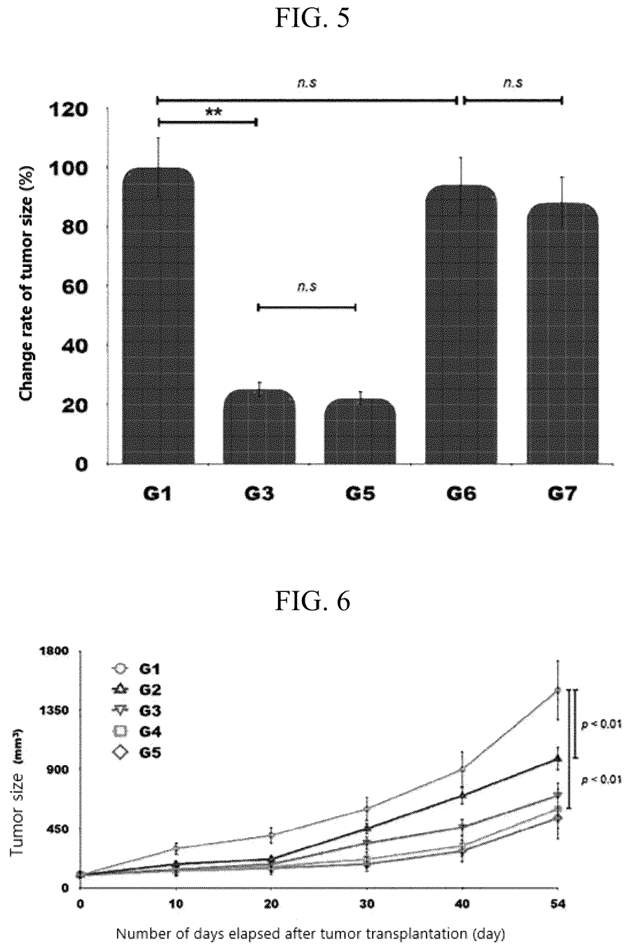

The results were evaluated by the level of apoptosis for each individual, and shown in Table 4 below, the tumor size according to the number of days elapsed after tumor transplantation is shown in Table 4, the tumor size according to the number of days elapsed after tumor transplantation is shown graphically in FIG. 4, and the relative amount of size change at the time of final biopsy in the transplantation model is shown in FIG. 5. For the measurement of the relative amount of size change at the time of final biopsy, the amount of change in G1 was considered as 100(%) among the average amount of size change during the experiment period for each group (G1, G3, G5, G6, G7), and the amount of change in each group was converted into a relative value, and shown in a graph as in Test Example 2-1.

Consistent with the results from the xenograft model for biliary tract carcinoma, the group in which NK cells cultured using alloferon was administrated showed a more excellent tumor growth inhibition effect than the positive control group. In particular, even when administered at the same number of cells, NK cells (G3) cultured using alloferon showed a remarkably superior tumor growth inhibition effect as compared to cells cultured in general medium (G6) or cells cultured in general NK medium (G7).

TABLE 4

| | | Proportion of | Level of apoptosis | | | |
|---|---|---|---|---|---|---|
| | N | death signal | for each individual | | | |
| Group | number | positive cells (%) | − | + | ++ | +++ |
| G1 | 8 | 7.3 | 8 | 0 | 0 | 0 |
| | | (SD = 2.1) | | | | |
| G2 | 8 | 13.2 | 1 | 3 | 4 | 0 |
| | | (SD = 4.2) | | | | |
| G3 | 8 | 33.4 | 1 | 1 | 5 | 1 |
| | | (SD = 9.1) | | | | |
| G4 | 8 | 35.1 | 0 | 3 | 2 | 3 |
| | | (SD = 8.5) | | | | |
| G5 | 8 | 17.8 | 1 | 3 | 3 | 1 |
| | | (SD = 4.4) | | | | |

−: No change, +: Weak, ++: Moderate, +++: Severe

### 2-3 Breast Cancer

The same procedure as Test Example 2-1 was performed, but MDA-MB-231 cells (2×10⁷ cells/0.2 mL) were subcutaneously injected into each group (G1˜G5) consisting of 10 nude mice, and subjected to transplantation and engraftment. After that, the breast cancer transplantation model was performed in the same manner as in Test Example 2-1, but the injection volume was set to 0.2 ml. Fourteen days after the last injection, mice bearing tumors were euthanized, and evaluated.

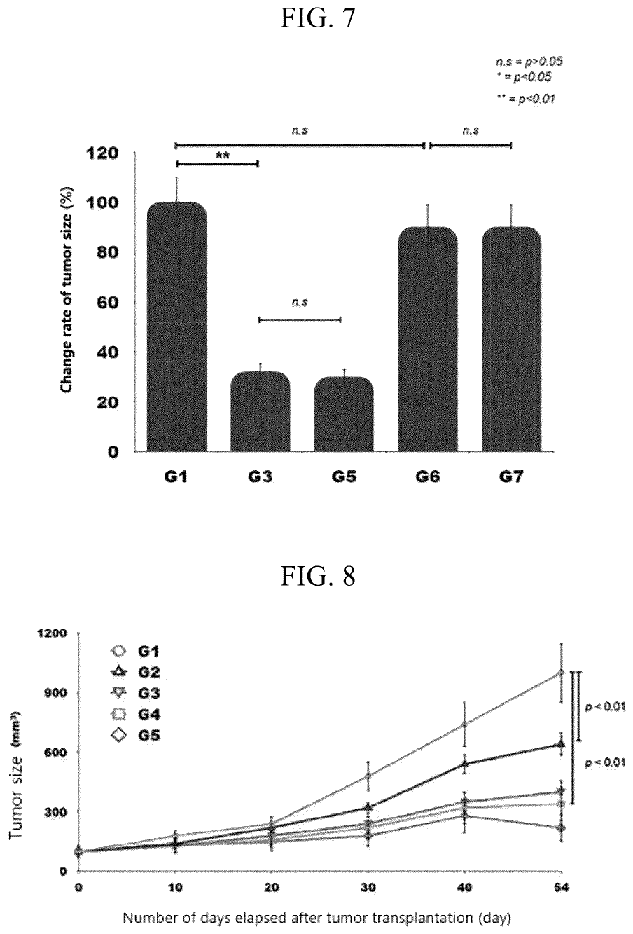

The results were evaluated by the level of apoptosis for each individual, and shown in Table 5 below, the tumor size according to the number of days elapsed after tumor transplantation is shown in FIG. 6, and the relative amount of size change at the time of final biopsy in the transplantation model is shown in FIG. 7. For the measurement of the relative amount of size change at the time of final biopsy, the amount of change in G1 was considered as 100(%) among the average amount of size change during the experiment period for each group (G1, G3, G5, G6, G7), and the amount of change in each group was converted into a relative value, and shown in a graph as in Test Example 2-1.

Consistent with the results from the xenograft model for biliary tract carcinoma and lung carcinoma, the group in which NK cells cultured using alloferon was administrated showed a more excellent tumor growth inhibition effect than the positive control group. In particular, even when administered at the same number of cells, NK cells (G3) cultured using alloferon showed a remarkably superior tumor growth inhibition effect as compared to cells cultured in general medium (G6) or cells cultured in general NK medium (G7).

TABLE 5

| | | Proportion of | Level of apoptosis | | | |
|---|---|---|---|---|---|---|
| | N | death signal | for each individual | | | |
| Group | number | positive cells (%) | − | + | ++ | +++ |
| G1 | 8 | 4.7 | 8 | 0 | 0 | 0 |
| | | (SD = 2.8) | | | | |
| G2 | 8 | 23.7 | 0 | 5 | 2 | 1 |
| | | (SD = 7.1) | | | | |
| G3 | 8 | 38.9 | 1 | 2 | 3 | 2 |
| | | (SD = 6.7) | | | | |
| G4 | 8 | 42.3 | 0 | 3 | 2 | 3 |
| | | (SD = 8.7) | | | | |
| G5 | 8 | 44.1 | 0 | 2 | 3 | 3 |
| | | (SD = 9.4) | | | | |

−: No change, +: Weak, ++: Moderate, +++: Severe

### 2-4 Prostate Cancer

The same procedure as Test Example 2-1 was performed, but P-3 cells (5×10⁶ cells/0.2 mL) were subcutaneously injected into each group (G1˜G5) consisting of 10 nude mice, and subjected to transplantation and engraftment. After that, the prostate cancer transplantation model was performed in the same manner as in Test Example 2-1, but the injection volume was set to 0.2 ml. Fourteen days after the last injection, mice bearing tumors were euthanized, and evaluated.

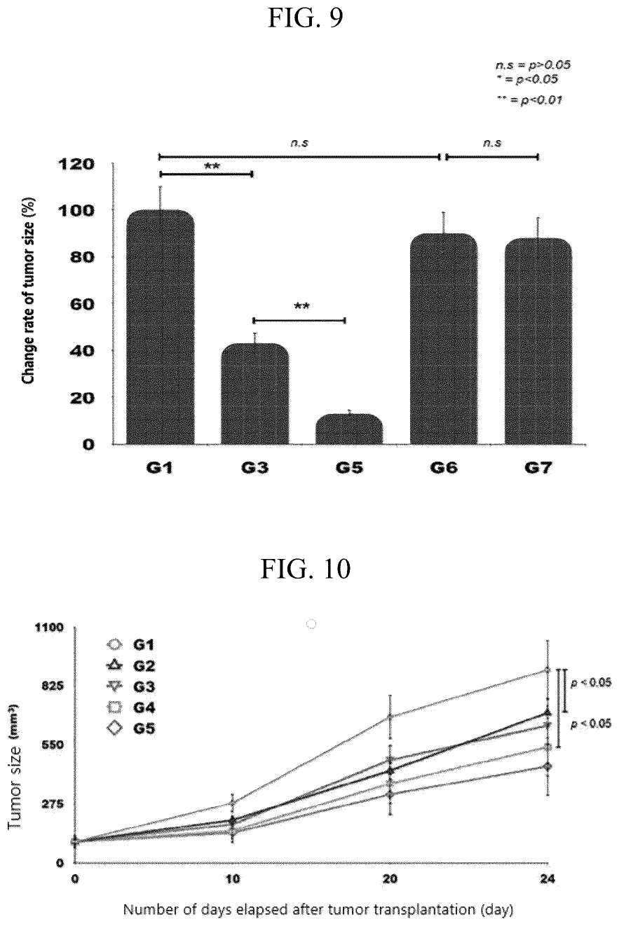

The results were evaluated by the level of apoptosis for each individual, and shown in Table 6 below, the tumor size according to the number of days elapsed after tumor transplantation is shown in Table 8, and the tumor size according to the number of days elapsed after tumor transplantation is shown graphically in FIG. 9. For the measurement of the relative amount of size change at the time of final biopsy, the amount of change in G1 was considered as 100(%) among the average amount of size change during the experiment period for each group (G1, G3, G5, G6, G7), and the amount of change in each group was converted into a relative value, and shown in a graph as in Test Example 2-1.

Consistent with the results from the xenograft model for biliary tract carcinoma, lung carcinoma and breast carcinoma, the group in which NK cells cultured using alloferon was administrated showed a more excellent tumor growth inhibition effect than the positive control group. In particular, even when administered at the same number of cells, NK cells (G3) cultured using alloferon showed a remarkably superior tumor growth inhibition effect as compared to cells cultured in general medium (G6) or cells cultured in general NK medium (G7).

TABLE 6

| | | Proportion of | Level of apoptosis | | | |
|---|---|---|---|---|---|---|
| | N | death signal | for each individual | | | |
| Group | number | positive cells (%) | − | + | ++ | +++ |
| G1 | 8 | 7.1 | 8 | 0 | 0 | 0 |
| | | (SD = 3.4) | | | | |
| G2 | 8 | 18.9 | 0 | 5 | 3 | 0 |
| | | (SD = 11.3) | | | | |
| G3 | 8 | 25.7 | 0 | 3 | 3 | 2 |
| | | (SD = 7.1) | | | | |
| G4 | 8 | 39.4 | 0 | 2 | 3 | 3 |
| | | (SD = 10.3) | | | | |
| G5 | 8 | 37.5 | 0 | 3 | 2 | 3 |
| | | (SD = 6.3) | | | | |

−: No change, +: Weak, ++: Moderate, +++: Severe

### 2-5. Melanoma

The same procedure as Test Example 2-1 was performed, but B16F10 cells (5×10⁶ cells/0.2 mL) were subcutaneously injected into each group (G1˜G5) consisting of 10 nude mice, and subjected to transplantation and engraftment. After that, the melanoma transplantation model was performed in the same manner as in Test Example 2-1, but the injection volume was set to 0.2 ml. In the case of melanoma, the 30-day survival rate after transplantation was low, and so the mice were sacrificed on the 24th, four days after D20. Mice bearing tumor were euthanized 4 days after the last injection, and then evaluated.

The results were evaluated by the level of apoptosis for each individual, and shown in Table 7 below, the tumor size according to the number of days elapsed after tumor transplantation is graphically shown in FIG. 10, and the relative amount of size change at the time of final biopsy in the transplantation model is shown in FIG. 11. For the measurement of the relative amount of size change at the time of final biopsy, the amount of change in G1 was considered as 100(%) among the average amount of size change during the experiment period for each group (G1, G3, G5, G6, G7), and the amount of change in each group was converted into a relative value, and shown in a graph as in Test Example 2-1.

Consistent with the results from the xenograft model for carcinoma previously performed, the group in which NK cells cultured using alloferon was administrated showed a more excellent tumor growth inhibition effect than the positive control group. In particular, even when administered at the same number of cells, NK cells (G3) cultured using alloferon showed a remarkably superior tumor growth inhibition effect as compared to cells cultured in general medium (G6) or cells cultured in general NK medium (G7).

TABLE 7

| | | Proportion of | Level of apoptosis | | | |
|---|---|---|---|---|---|---|
| | N | death signal | for each individual | | | |
| Group | number | positive cells (%) | − | + | ++ | +++ |
| G1 | 8 | 16.3 | 6 | 1 | 1 | 0 |
| | | (SD = 4.3) | | | | |
| G2 | 8 | 47.5 | 1 | 3 | 3 | 1 |
| | | (SD = 8.3) | | | | |
| G3 | 8 | 64.4 | 1 | 1 | 5 | 1 |
| | | (SD = 7.4) | | | | |
| G4 | 8 | 68.1 | 0 | 2 | 3 | 3 |
| | | (SD = 10.5) | | | | |
| G5 | 8 | 34.8 | 0 | 4 | 3 | 1 |
| | | (SD = 8.1) | | | | |

−: No change, +: Weak, ++: Moderate, +++: Severe

### 2-6. Pancreatic Cancer

The same procedure as Test Example 2-1 was performed, but Capan-2 cells (1×10⁷ cells/0.2 mL) were subcutaneously injected into each group (G1˜G5) consisting of 10 nude mice, and subjected to transplantation and engraftment. After that, the pancreatic cancer transplantation model was performed in the same manner as in Test Example 2-1, but the injection volume was set to 0.2 ml. In the case of pancreatic cancer, the 30-day survival rate after transplantation was low, and so the mice were sacrificed on the 24th, four days after D20. Mice bearing tumor were euthanized 4 days after the last injection, and then evaluated.

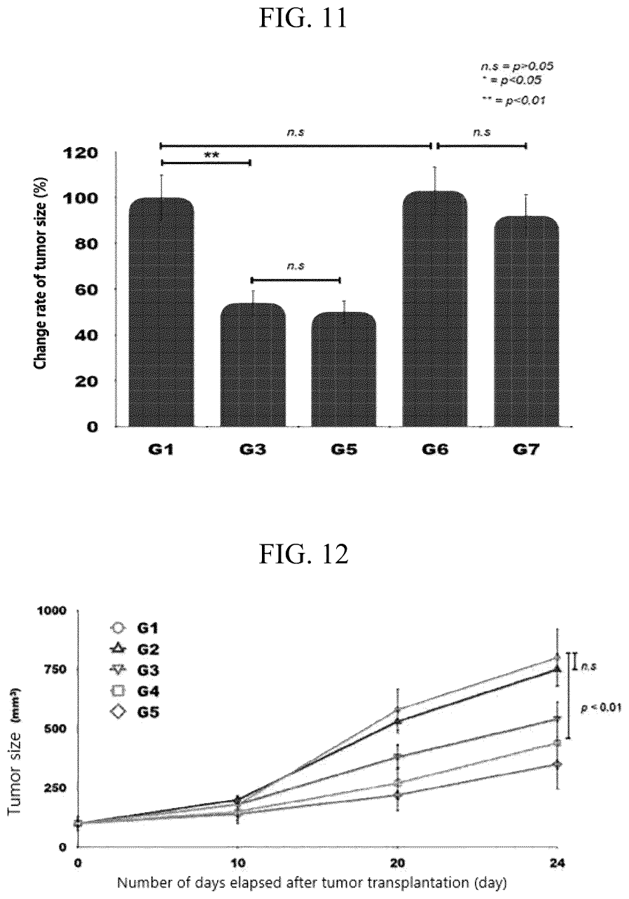

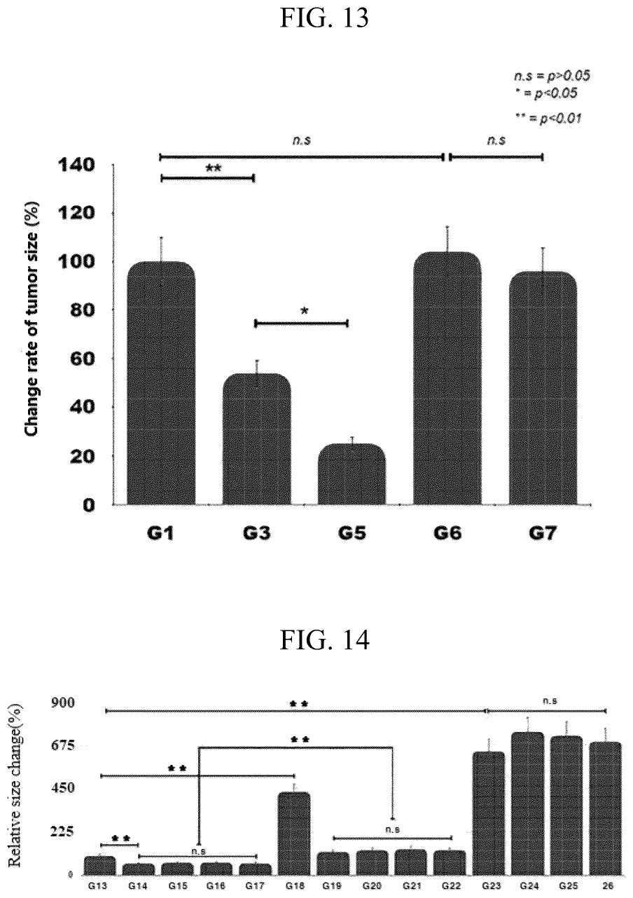

The results were evaluated by the level of apoptosis for each individual, and shown in Table 8 below, the tumor size according to the number of days elapsed after tumor transplantation was shown in FIG. 12, and the relative amount of size change at the time of final biopsy in the transplantation model is shown in FIG. 13. For the measurement of the relative amount of size change at the time of final biopsy, the amount of change in G1 was considered as 100(%) among the average amount of size change during the experiment period for each group (G1, G3, G5, G6, G7), and the amount of change in each group was converted into a relative value, and shown in a graph as in Test Example 2-1.

Consistent with the results from the xenograft model for carcinoma previously performed, the group in which NK cells cultured using alloferon was administrated showed a more excellent tumor growth inhibition effect than the positive control group. In particular, even when administered at the same number of cells, NK cells (G3) cultured using alloferon showed a remarkably superior tumor growth inhibition effect as compared to cells cultured in general medium (G6) or cells cultured in general NK medium (G7).

TABLE 8

| | | Proportion of | Level of apoptosis | | | |
|---|---|---|---|---|---|---|
| | N | death signal | for each individual | | | |
| Group | number | positive cells (%) | − | + | ++ | +++ |
| G1 | 8 | 4.7 | 8 | 0 | 0 | 0 |
| | | (SD = 1.2) | | | | |
| G2 | 8 | 13.5 | 2 | 3 | 3 | 0 |
| | | (SD = 4.3) | | | | |
| G3 | 8 | 27.1 | 1 | 2 | 4 | 1 |
| | | (SD = 8.7) | | | | |
| G4 | 8 | 33.4 | 0 | 1 | 4 | 3 |
| | | (SD = 6.2) | | | | |
| G5 | 8 | 35.2 | 0 | 4 | 2 | 2 |
| | | (SD = 5.4) | | | | |

−: No change, +: Weak, ++: Moderate, +++: Severe

### Test Example 3: Confirmation of Tumor Inhibitory Effect Depending on the Type of Alloferon

NCr athymic nude mice (BALB/cSlc-nu/nu) (Charles River Laboratories) were manipulated and raised in a microisolator cage according to the Laboratory Practice for Nonclinical Laboratory Studies (21CFR Part 58, Food and Drug Administration, United States of America) (Apr. 1, 2015). H460 cells (5×10⁶ cells/0.2 mL) were subcutaneously injected into each group (G13˜G16) consisting of 10 nude mice, and subjected to transplantation and engraftment. In each group, eight mice with well-engrafted tumors with a volume of 84˜119 mm³ (body weight range of 19.3˜20.5 g) were selected and then subjected to medical treatments. NK cells were injected intravenously three times at 10-day intervals into nude mice bearing H460 tumors. As a positive control group, gemcitabine (Gem) and cis-diamineplatinum(II) dichloride (CDDP) were administered at doses of 120 mg/kg and 3 mg/kg, respectively. When injecting NK cells, cells were injected intravenously into three groups of mice, and alloferon sequences 1 to 4 were added to a physiological saline solution as a vehicle for each group at a concentration of 10 mg/L, and changes in the size of lung cancer were confirmed.

The number of cells administered to each group and the alloferon treatment method are shown in Table 10 below.

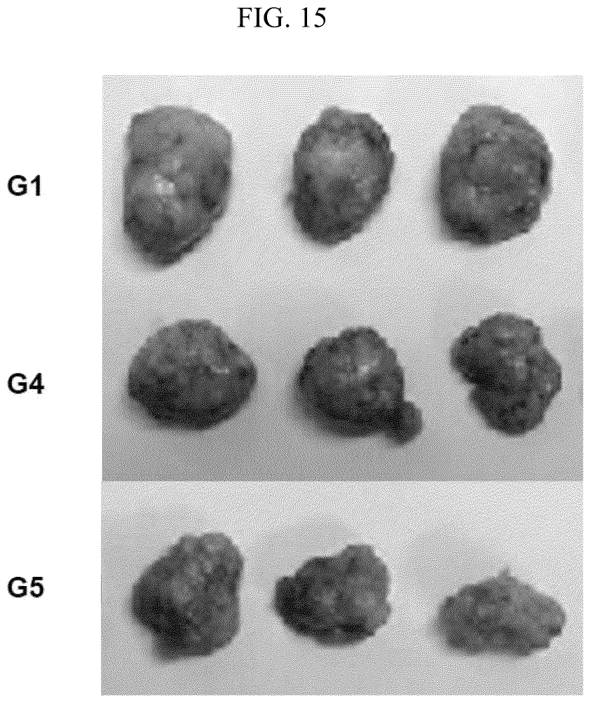

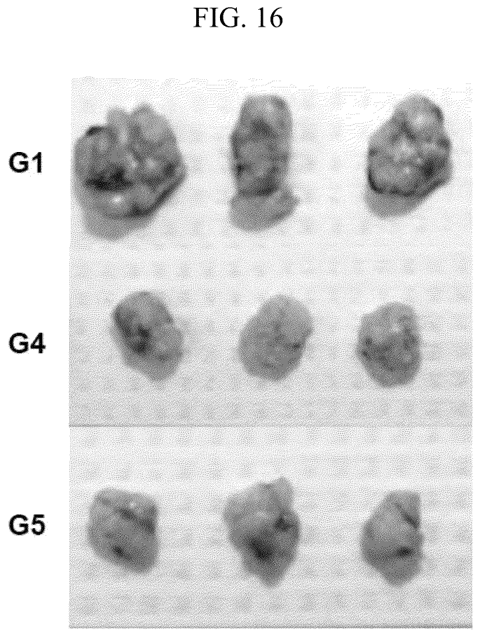

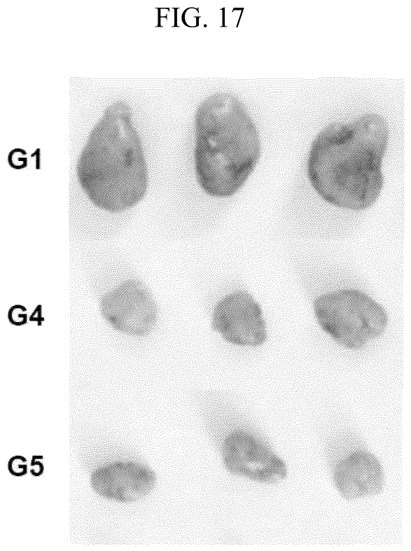

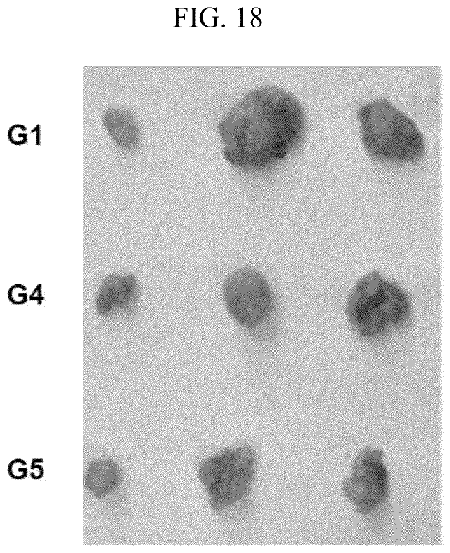

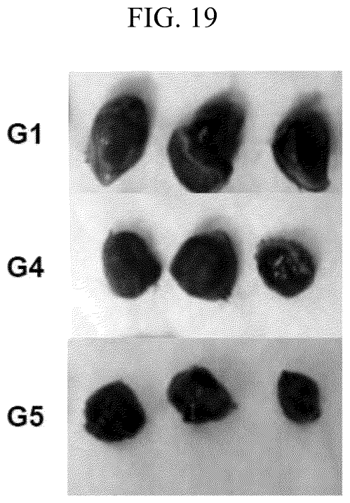

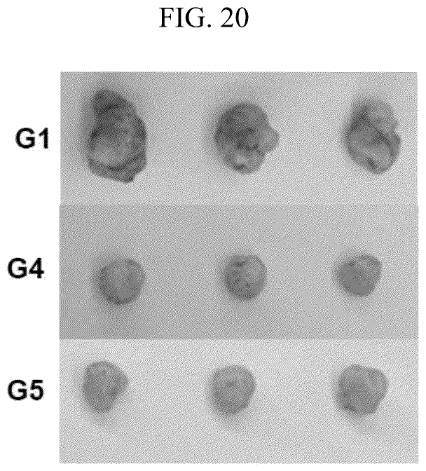

The results are shown in FIG. 15. IT could be confirmed that when NK cells were cultured, a tumor growth inhibitory effect was observed when alloferon was treated and when NK cells cultured in general NK cell culture medium and alloferon were simultaneously administered, whereas when only alloferon was administered without treating NK cells, or when NK cells are treated but not with alloferon, the tumor growth inhibitory effect is significantly slowed.

TABLE 10

| | Cell count | | Concentration | |
|---|---|---|---|---|
| Group | (cells/ | | of alloferon | Treatment |
| name | 0.2 ml) | Description | co-treatment | alloferon |
| G13 | 1 × 10⁶ | Cells cultured | 0 | Untreated |
| G14 | | under NK | 10 mg/L | Alloferon 1 |
| G15 | | medium + | | Alloferon 2 |
| G16 | | Alloferone 1 | | Alloferon 3 |
| G17 | | (10 mg/L) | | Alloferon 4 |
| G18 | | conditions | 0 | Untreated |
| | | (medium dose) | | |
| G19 | | Cells cultured | 10 mg/L | Alloferon 1 |
| G20 | | under NK | | Alloferon 2 |
| G21 | | medium conditions | | Alloferon 3 |
| G22 | | (medium dose) | | Alloferon 4 |
| G23 | 0 | Cell untreated | 10 mg/L | Alloferon 1 |
| G24 | | | | Alloferon 2 |
| G25 | | | | Alloferon 3 |
| G26 | | | | Alloferon 4 |

### Industrial Applicability

The present invention has industrial applicability in the anticancer treatment industry.

### SEQUENCE LIST FREE TEXT

<110>NK Medics
<120>Composition for preventing or treating cancer containing NK cells cultured using alloferon
<130>PP220004
<160>4
<170>KoPatentIn 3.0
<210>1
<211>13
<212>PRT
<213>Artificial Sequence
<220>
<223>engineered from known peptides
<400>1
His Gly Val Ser Gly His Gly Gin His Gly Val His Gly
1 5 10
<210>2
<211>12
<212>PRT
<213>Artificial Sequence
<220>
<223>engineered from known peptides
<400>2
Gly Val Ser Gly His Gly Gln His Gly Val His Gly
1 5 10
<210>3
<211>8
<212>PRT
<213>Artificial Sequence
<220>
<223>engineered from known peptides
<400>3
Ser Gly His Gly Gln His Gly Val
1 5
<210>4
<211>10
<212>PRT
<213>Artificial Sequence
<220>
<223>engineered from known peptides
<400>4
Val Ser Gly His Gly Gln His Gly Val His
1 5 10

1. A composition for preventing or treating cancer comprising NK cells cultured in the presence of a polypeptide represented by the amino acid sequence of SEQ ID NOs: 1-4 or a combination thereof.

2. The composition for preventing or treating cancer according to claim 1, wherein the NK cells are cultured in a medium containing 6 mg/L to 40 mg/L of the polypeptide or a combination thereof.

3. The composition for preventing or treating cancer according to claim 1, wherein the NK cells are cultured in a medium containing a polypeptide represented by any one of the amino acid sequences of SEQ ID NOs: 1-4 or a combination thereof; and IL-2, IL-12, IL-15, OKT-3, or a combination thereof.

4. The composition for preventing or treating cancer according to claim 1, wherein the cancer is a solid cancer.

5. The composition for preventing or treating cancer according to claim 1, wherein the solid cancer is selected from the group consisting of biliary tract cancer, lung cancer, breast cancer, prostate cancer, melanoma, and pancreatic cancer.

6. The composition for preventing or treating cancer according to claim 1, wherein the NK cells are contained at a density of 2.5×10⁵ cell/ml or more.

7. A composition for preventing or treating cancer comprising a polypeptide represented by the amino acid sequence of SEQ ID NOs: 1-4 or a combination thereof; and NK cells.

8. The composition for preventing or treating cancer according to claim 7, wherein the composition contains 10 μg or more of the polypeptide represented by the amino acid sequence of SEQ ID NOs: 1 to 4 or a combination thereof based on 1×10⁶ cells of NK cells.
